(19) United States
(12) Patent Application Publication
(10) Pub. No.: US 2018/0258091 A1
(43) Pub. Date: Sep. 13, 2018

(54) PROCESS FOR MANUFACTURE OF FORODESINE

(71) Applicant: EURO-CELTIQUE S.A, Luxembourg (LU)

(72) Inventors: Francis BASS, Avoca (IE); Richard LYONS, Arklow (IE); Nicholas REILLY, Westport (IE)

(21) Appl. No.: 15/542,120

(22) PCT Filed: Jan. 7, 2016
(86) PCT No.: PCT/EP2016/050191

§ 371 Date: Jul. 7, 2017

(30) Foreign Application Priority Data
Jan. 7, 2015 (GB) 1500216.5

(51) Int. Cl. C07D 487/04

(52) U.S. Cl. CPC C07D 487/04

(57) ABSTRACT

This invention describes a novel process for the manufacture of Forodesine (I).

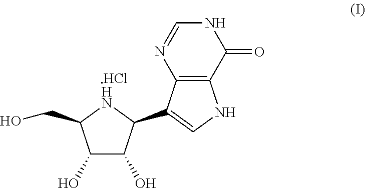 [SMILES: O=c1[nH]cnc2c([C@@H]3N[C@H](CO)[C@@H](O)[C@H]3O)c[nH]c12 ; ".HCl"]

### FIELD OF THE INVENTION

The invention describes a new process for the manufacture of Forodesine.

### BACKGROUND

Forodesine, or 7-[(2S,3S,4R,5R)-3,4-dihydroxy-5-(hydroxymethyl)-2-pyrrolidinyl]-1,5-dihydropyrrolo[2,3-e]pyrimidin-4-one, is an inhibitor of purine nucleoside phosphorylase. It is currently in development as a treatment for peripheral T-Cell Lymphoma.

WO99/19338 describes a compound genus as a new class of inhibitors of nucleoside metabolism, including Forodesine. The compounds effect as inhibitors of purine nucleoside phosphorylase is taught as efficacious to suppress T-cell function and to treat infections caused by protozoan parasites.

WO00/61783 describes a number of processes for preparing molecules described in WO99/19338. Reaction scheme 3 on page 23 of the published application describes a synthesis of Forodesine, characterised by the removal of two acid labile protecting groups in the final step to yield the hydrochloride salt.

Forodesine is a particularly difficult molecule to make on a commercial scale. The current process for manufacture requires a coupling reaction under cryogenic temperature conditions of −55 C. Subsequent steps involve the use of a high pressure hydrogenation reaction. Such extreme reaction conditions provide for safety concerns, particularly when conducted on a bulk scale. Further the products of the reaction were extremely challenging to purify. The effect of all this is to require more sophisticated and expensive equipment at the manufacturing plant; all of which add up to an increased cost of goods for patients. Accordingly a new manufacturing process was sought.

Surprisingly a new route has been invented which is shorter, cheaper, less dangerous and provides an increased overall yield whilst still conforming to the required purity profile.

The current manufacturing process is described in FIG. 1.

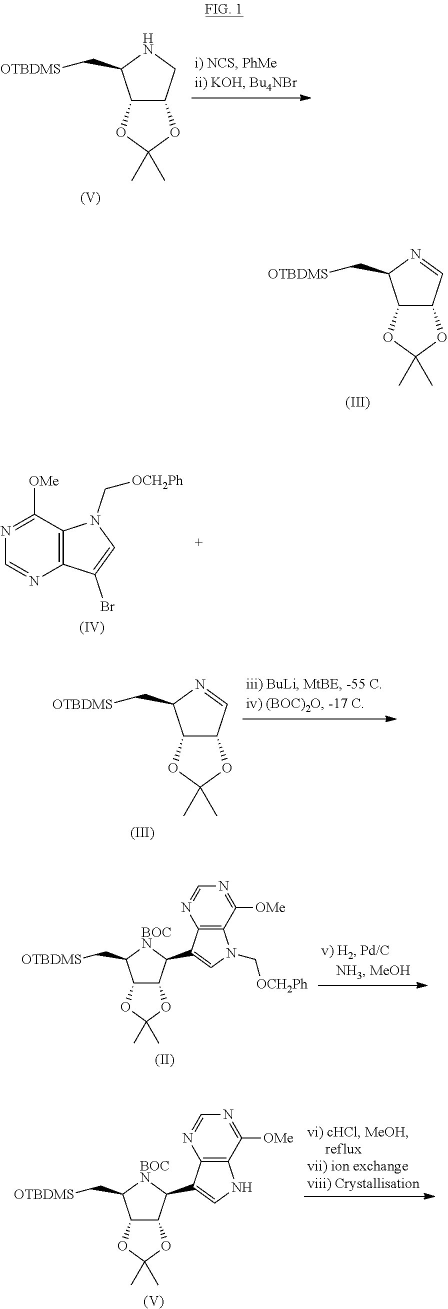

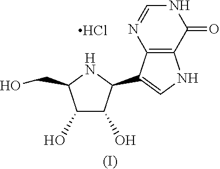

Within the diagram, the following acronyms are used, wherein NCS is N-Chlorosuccinimide, OTBDMS is t-butyldimethylsiloxy protecting group, MtBE is methyl t-butyl ether, (BOC)₂O is di-t-butyldicarbonate and BOC is t-butyloxycarbonyl protecting group,

Particularly problematic in this process is the requirement to conduct the coupling of process step (iii) at exceptionally low temperature. Further challenges are provided by process step (v) the hydrogenation reaction to remove the benxylyoxymethyl (BOM) protecting group, before removing the other acid labile protecting groups.

Conducting hydrogenation reactions with their need for a high pressure environment requires specialist equipment. Such apparatus is expensive, adding to the cost of the materials produced. Despite the use of specialist equipment, safety concerns can never be eradicated. Whilst BOM can, in certain circumstances, be acid labile, treatment of analogues of the molecules described in FIG. 1 with acid has always resulted in incomplete removal of the protecting group, leading to a large number of partially deprotected impurities. This makes purification exceptionally difficult as well as reducing the overall yield for the step.
A new improved process has been developed as described in FIG. 2:

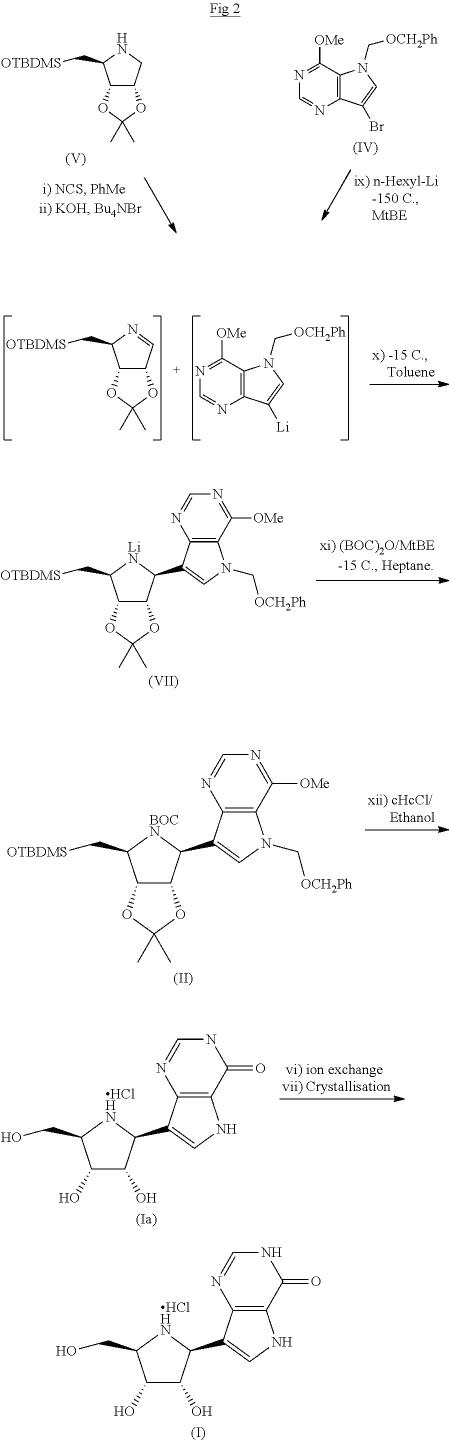

The new route has a number of clear advantages. The coupling reaction (ix) is conducted at a warmer −15° C., rather than the challenging cryogenic conditions of −55° C. required previously. It eradicates the hydrogenation step, avoiding the need for dangerous high pressure conditions. It also makes the overall process much quicker and cheaper; not only are the conditions challenging, but the reagents used in large quantities such as palladium are expensive and environmentally challenging.

The classical method to remove a BOM protecting group is by catalytic hydrogenation. It is however known to be unstable in acid conditions. For this reasons there have been previous attempts to remove BOM at the same time as the three acid labile protecting groups. This has always been unsuccessful as treatment with acid typically resulted in incomplete deprotection, leading to a mixture of products. This made for a tricky purification and a reduced yield. Surprisingly under the particular conditions described herein it has been possible to effect the transformation in greater yield and without a difficult purification. The final product is obtained in equal or greater purity than material obtained from the previous route.

The present invention provides for:

A process for the manufacture of a compound of Formula (I)

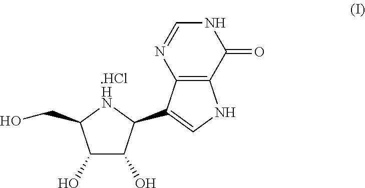 [SMILES: O=c1[nH]cnc2c([C@@H]3N[C@H](CO)[C@@H](O)[C@H]3O)c[nH]c12 ; ".HCl"]

Comprising treating a compound of formula (II)

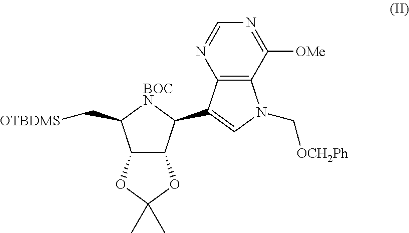

With concentrated acid.

Preferably the acid is concentrated hydrochloric acid.

In one embodiment the compound of formula (II) is treated with conc hydrochloric acid (conc HCl) in ethanol for 16 hours before being heated to 40° C. for a further 8 hours.

In further embodiments of the invention, the reaction mixture is heated to 90-100° C. for a period, prior to isolation of the reaction product.

In a further embodiment of the invention, the reaction mixture is treated with ammonium hydroxide prior to isolation of the reaction product.

In a preferred embodiment the compound of formula (II) is treated with conc hydrochloric acid (conc HCl) in ethanol for 16-24 hours at room temperature.

In a further preferred embodiment the reaction product is purified by ion exchange and recrystallization from ethanol.

Preferred recrystallisation conditions are to dissolve the Forodesine product in dilute aqueous HCl at elevated temperature. Suitable temperatures are well known to the person skilled in the art. In one embodiment, a temperature of 45 C is used. The solution is cooled to 20° C. and ethanol added over at least 1 h. The mixture is then seeded with Forodesine HCl. The resulting slurry is stirred for 8 h at 20° C., then cooled to 2° C. for a further 1.5 h. The product is isolated by filtration, washed twice with cold ethanol then dried.

Suitable ion exchangers are well known to those skilled in the art and include the Dowex 50WX4 resin in the Na⁺ form.

The invention also provides for the synthesis of a compound of formula (II)

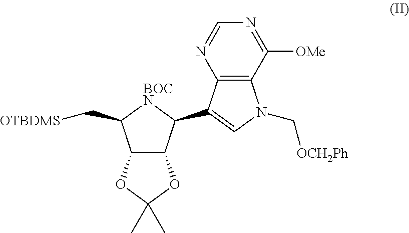

By reacting a compound of Formula (VII)

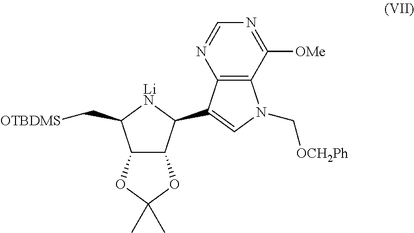

With di-t-butyldicarbonate.
Preferably the reaction is conducted at −10 to −20° C., in methyl t-butyl ether & heptane

The invention also provides for the synthesis of a compound of formula (VII)

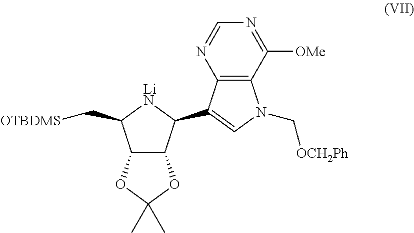

By reacting a compound of Formula (IV)

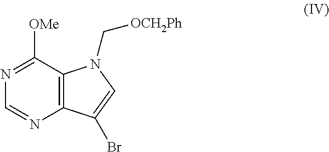

With a suitable base to form

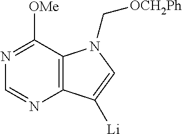

Before reacting with a compound of Formula (III)

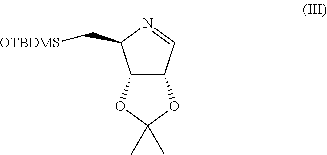 [SMILES: CC1(C)O[C@H]2[C@H](C=N[C@@H]2CO[Si](C)(C)C(C)(C)C)O1]

in a suitable solvent at a temperature of 0 to −50° C.

Suitable bases include alkyl lithium reagents such as butyl lithium or hexyl lithium. Preferably the base is hexyl lithium.

Suitable solvents include toluene and methyl t-butyl ether

Preferred temperature range for the reaction is −5 to −45° C.

More preferably the temperature range is −10 to −20° C.

Most preferably temperature is −15 to −17° C.

Starting materials (III) and (IV) may be obtained by the synthetic routes described within WO00/61783.

### EXAMPLES

All reagents were obtained from the Sigma-Aldrich company Ltd.

### Example 1

### Stage 1 Manufacture of (III)

Compound of formula (III) (approx. 130 g) in toluene solution is added to a suspension of N-Chlorosuccinimide in toluene at 20° C. over a period of 90 min. The reaction mixture is stirred at 20° C. for 1 hour then chilled to 0° C. and stirred for a further hour. The precipitated succinimide by-product is removed by filtration and the filtered solution charged directly to a 45% potassium hydroxide solution (aq) containing tetrabutylammonium bromide. The reaction mixture is stirred at 0° C. and completion of reaction is confirmed by GC analysis. Water is then added to the two-phase mixture to dissolve inorganic precipitates and the toluene product solution is washed with a 28% ammonium hydroxide/acetic acid buffer mixture with sodium chloride added. After phase separation the organic phase solution is stabilised with triethylamine. Magnesium sulfate is added to dry the solution. After filtration, the yield of (III) is determined by R.O.E. and GC purity.

### Stage 2 Manufacture of (II)

### Stage 2a Lithiation

A suspension of compound of formula (IV) (approx. 200 g) in MtBE is chilled to −15° C. and treated with n-Hexyl lithium (2.5M in hexanes) added over 2 h, maintaining the reaction mixture at −15° C. The mixture is then stirred for 3 h at −15° C.

Stage 2b Coupling with (IV)

After lithiation is complete, a compound formula (III) in toluene solution is added to the reaction mixture maintaining the contents at −15° C. The reaction mixture is then stirred at this temperature for 1.5 h.

### Stage 2c Boc Anhydride Quench

A solution of di-t-butyldicarbonate in MtBE is added to the above reaction mixture at −15° C. The solution is stirred for a further 30 min.

### Workup and Purification

The reaction mixture is quenched by addition of RO water, then filtered. The aqueous layer is separated and run to waste. The organic layer is again washed with water. The organic layer is concentrated to a low volume and solvent replaced by heptane. The mix is stirred for 16 h and filtered again.

The solution is passed through a silica gel column and eluted with heptane. The resulting solution is treated with charcoal—stirred for 3 h, then filtered. The product (II) is progressed as a solution in heptane to the next stage.

### Stage 3 Manufacture of Crude Forodesine (Ia)

Stage 3 Deprotection with Conc. HCl

Concentrated hydrochloric acid is added to (II) in heptane and the mixture stirred. The acid phase is separated off and stirred for 16 h at ambient temperature. The solution is then heated to 40° C. for 6 h. The water is then distilled off under reduced pressure to a minimum volume.

Ethanol is then added to precipitate the crude Forodesine (Ia) which is isolated by filtration after cooling 0-5° C. It is washed with ethanol and dried in a vacuum oven at 75° C. to a constant weight.

### Stage 4a Decolourization of Crude Forodesine (Ia) Using Ion-Exchange Column

Crude Forodesine (Ia) is dissolved in water and loaded onto a freshly prepared ion-exchange column containing Dowex 50WX4 resin in the Na⁺ form activated with 30% sodium hydroxide solution. The ion-exchange column is eluted with 4×100 mL water followed by 4×100 mL 2M HCl. The HCl fractions are collected separately as they contain the desired product. The 2M HCl fractions are combined and concentrated under vacuum with minimum RO water added to dissolve the residue. 1,4-Dioxane is added to the aqueous solution to precipitate the product. The mixture is stirred at 20° C. for 1.5 h. The product is filtered, washed with 1,4-dioxane and dried in a vacuum oven at 35° C. to a constant weight to give decolourised BCX1777.

### Stage 4b Recrystallization of Forodesine

Decolourised Forodesine is added to in 0.6M dilute hydrochloric acid and heated to 45° C. to dissolve. The resulting solution is hot filtered and washed through with some RO Water. The solution is cooled to 20° C. and ethanol added over at least 1 h. The mixture is then seeded with Forodesine HCl. The resulting slurry is stirred for 8 h at 20° C., then cooled to 2° C. for a further 1.5 h. The product is isolated by filtration, washed twice with cold ethanol then dried in a vacuum oven at 75° C. to a constant weight to give a white crystalline Forodesine HCl (approx. 50 g).

While the invention has been described in detail and with reference to specific embodiments thereof, it will be apparent to one of skill in the art that various changes and modifications can be made therein without departing from the spirit and scope thereof. Moreover, all embodiments described herein are considered to be broadly applicable and combinable with any and all other consistent embodiments, as appropriate.

1. A process for the manufacture of a compound of Formula (I) 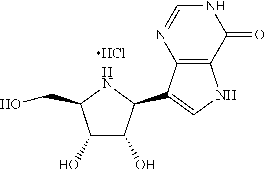 [SMILES: O=c1[nH]cnc2c([C@@H]3N[C@H](CO)[C@@H](O)[C@H]3O)c[nH]c12 ; Cl]
Comprising treating a compound of formula (II) 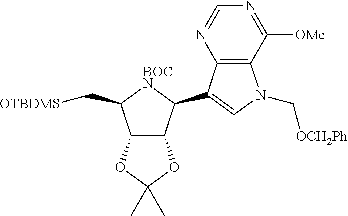
With concentrated acid

2. The process as claimed in claim 1 where the acid is concentrated hydrochloric acid.

3. The process as claimed in claim 1, where compound of formula (II) is treated with concentrated hydrochloric acid for 16-24 hours at room temperature.

4. A process as claimed in claim 1 with the additional step of obtaining a compound of formula (II) 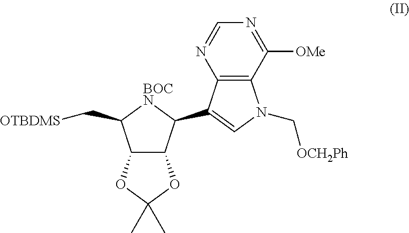
By reacting a compound of Formula (VII) 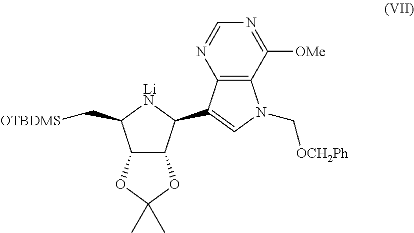
With di-t-butyldicarbonate.

5. The process as claimed in claim 4 wherein the reaction is conducted at −10 to −20° C.

6. A process as claimed in claim 4 wherein a compound of formula (VII) 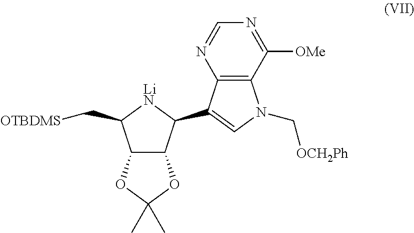
Is obtained by reacting a compound of Formula (IV) 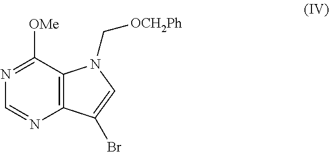
With a suitable base to form 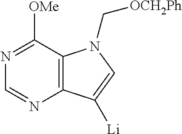
Before reacting with a compound of Formula (III) 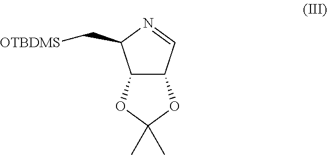 [SMILES: CC1(C)O[C@H]2[C@H](C=N[C@@H]2CO[Si](C)(C)C(C)(C)C)O1]
At 0 to −50° C. in a suitable solvent.

7. Process as claimed in claim 6 where the base is hexyl lithium.

8. Process as claimed in claim 6 where the temperature range is −10 to −20° C.
